(19) United States
(12) Patent Application Publication
(10) Pub. No.: US 2007/0154528 A1
(43) Pub. Date: Jul. 5, 2007

(54) Flexible wound dressing

(75) Inventors: Eckart Mathias, Goleta, CA (US); Robert Ibsen, Santa Maria, CA (US)

Correspondence Address: CROWELL & MORING LLP;INTELLECTUAL PROPERTY GROUP, P.O. BOX 14300, WASHINGTON, DC, 20044-4300, US

(73) Assignee: Den-Mat Corporation

(21) Appl. No.: 11/637,665

(22) Filed: Dec. 13, 2006

Related U.S. Application Data
(60) Provisional application No. 60/749,605, filed on Dec. 13, 2005.

(51) Int. Cl. A61K 9/70

(52) U.S. Cl. 424/443; 602/48; 424/445; 424/603

(57) ABSTRACT

The present invention relates to flexible wound dressings. More particularly, the present invention is directed to flexible wound dressings comprising ionomer resins. The invention involves treating the injured tissue with films comprising a special biocompatible crosslinked resin that optionally contains leachable healing and antibiotic agents.

### FIELD OF THE INVENTION

The present invention relates to flexible wound dressings. More particularly, the present invention is directed to flexible wound dressings comprising ionomer resins.

### BACKGROUND OF THE INVENTION

The invention relates to wound dressings for aiding tissue repair by promoting the growth of skin tissue with which they are in contact. The invention involves treating the injured tissue with films comprising a special biocompatible crosslinked resin that optionally contains leachable healing and antibiotic agents.

Early materials used to treat skin wounds included medicated and unmedicated cotton wools, gauzes, tows, and lints; gauze and cotton tissues, bandages, jaconet, oiled silk and emplastrums. From 1960 on, a new generation of products was developed based on the realization that the control of micro-environments was necessary if wound healing was to progress to the optimum degree.

Advances in the development of synthetic polymers produced the most radical changes in wound care dressings as factors such as water vapor, oxygen permeability, bacterial impermeability, and selective absorption could be incorporated into new formulations along with specific requirements such as conformability, non-adherence, and adhesiveness. This family of polymeric products included polymeric foams, polymeric films, particulate and fibrous polymers, hydrogels and hydrocolloids.

Hydrogels are three dimensional cross-linked networks of hydrophilic polymers that are prepared from materials such as gelatin, polysaccharides, cross-linked polyacrylamide polymers, polyelectrolyte complexes, and polymers or copolymers derived from methacrylate esters. These interact with aqueous solutions by swelling to an equilibrium value and retaining a significant proportion of water within their structure. They are insoluble in water.

In contrast to the single polymer hydrogels described, the products designated as hydrocolloids are complex formulations that contain not only colloids but elastomeric and adhesive components. Hydrocolloids have an adhesive formulation that gives an initial adhesion higher than some surgical adhesive tapes. After application, the absorption of transepidermal water vapor will modify the adhesive flow to maintain a high tack and adhesive performance throughout the period of use.

A common problem in the management of both acute and chronic wounds is the maintenance of an optimal level of moisture over the wound bed during heavy exudate drainage. This is usually, but not always, an early stage of healing. Most moist wound dressing technologies such as films, hydrocolloid dressings and hydrogels are typically overwhelmed by the accumulated exudate moisture during this heavy drainage phase. Management of moisture during heavy exudate drainage often necessitates the use of gauze or sponge packings that wick away excess moisture from the wound bed, thin film coverings that trap exudate fluid over the wound bed, or calcium alginate dressings that chemically bind exudate moisture due to the hygroscopic properties of the seaweed extract.

Known hydrocolloid dressings are subject to a number of drawbacks. The major disadvantages of these dressings include the potential to disintegrate in the presence of excess fluid at the wound site, and minimal, virtually negligible, control over water loss from the wound. This latter disadvantage is particularly important, as excess water loss from a wound will cause an increase in heat loss from the body as a whole, potentially leading to hypermetabolism. In addition, hydrocolloid dressings require frequent dressing changes.

There has also been proposed the use of a biocompatible wound dressing based on fibrin. One mechanism for hemostasis, i.e., prevention of blood loss, of a mammal is the formation of a blood clot. Clot formation in humans, i.e., blood coagulation, occurs by means of a complex cascade of reactions with the final steps being the conversion of fibrinogen—a monomer—by thrombin, calcium ions and activated factor XIII to form ultimately crosslinked fibrin II polymer, which is the fibrin clot.

The formation of crosslinked fibrin II polymer proceeds by the fibrinogen being converted by thrombin to fibrin I monomer, which spontaneously polymerizes to form fibrin I polymer, which is sometimes referred to as soluble fibrin I because by treatment by appropriate chemical means the fibrin I polymer can be reconverted to fibrin I monomer. The fibrin I polymer is then converted by thrombin to fibrin II polymer, which is sometimes referred to as soluble fibrin II because by treatment by appropriate chemical means the fibrin II polymer can be converted to fibrin II monomer. The fibrin II polymer, under the influence of factor XIIIa—known as activated factor XIII—is then crosslinked to form crosslinked fibrin II, which is the fibrin clot. Factor XIII is activated by thrombin in the presence of calcium ions. Cross-linked fibrin II is sometimes referred to as insoluble fibrin II because it cannot be converted to fibrin II monomer.

Fibrinogen represents about 2 to 4 grams/liter of the blood plasma protein. Fibrinogen is a monomer that consists of three pairs of disulfide-linked polypeptide chains designated (α.)₂, (β.)_{.2}, _{γ.2.} “A” and “B” represent the two small aminoterminal peptides, known as fibrinopeptide A and fibrinopeptide B, respectively. The cleavage of fibrinopeptides A from fibrinogen in the transformation of fibrinogen by thrombin results in the fibrin I compound and the subsequent cleavage of fibrinopeptides B results in the fibrin II compound. Such cleavage of fibrinopeptides A and B reduces the molecular weight of fibrinogen by an extremely small amount, about 6,000 out of 340,000 daltons, but exposes the polymerization sites.

A fibrin sealant is a biological adhesive whose effect imitates the final stages of coagulation, thereby resulting in a fibrin clot. Conventional fibrin sealants consist of concentrated human fibrinogen, bovine aprotinin and factor XIII, as the first component and bovine thrombin and calcium chloride as the second component. Application is generally carried out with a double-barrelled syringe, which permits simultaneous application of both components to the site where one wants to form the fibrin clot. Aprotinin is a fibrinolytic inhibitor added to promote stability of fibrin sealants. U.S. Pat. No. 6,310,267, issued to Rapp, discloses a fibrin based wound covering with a biodegradable carrier support.

Wound dressings have also been combined with a biodegradable carrier material. Common carriers include natural or chemically modified collagen, keratin, gelatin, carbohydrates or cellulose derivatives. Synthetic, biodegradable polymer carriers have also been proposed. These include polyhydroxycarboxylic acids, polyesters, polycyanoacrylates, polyamino acids, polyalcohols and silicones. These carrier materials are commonly employed as a web or as a fabric.

Collagen carriers suffer from numerous deficiencies. Collagen films do not readily conform to varied wound shapes. Furthermore, some collagen wound dressings have poor fluid absorption properties and undesirably enhance the pooling of wound fluids.

Wound dressings have also been combined with numerous pharmacological and/or antibiotic compositions. Examples of such compositions include, but are not limited to, antifungal compositions, antiviral compositions, antibacterial compositions, and antiparasitic compositions. Examples of antimicrobial compositions that can be used in the present invention include, but are not limited to, isoniazid, ethambutol, clofazimine, rifabutin, fluoroquinolones, pyrazinamide, streptomycin, ofloxacin, ganciclovir, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, erythromycin, ciprofloxacin, clindamycin, lincomycin, acyclovir, trifluorouridine, pentamidine, atovaquone, paromomycin, diclazaril, acyclovir, trifluorouridine, foscarnet, penicillin, gentamicin and sparfloxacin.

Geristore® and Tenure®, sold by Den-Mat Corporation, Santa Maria, Calif., are promoted for certain uses in dentistry. U.S. Pat. Nos. 4,738,722, 5,334,625 and 5,151,453, incorporated herein by reference, describe Geristore®. Geristore® is a small particle composite that contains fluoride, is radiopaque and hydrophilic. It has low-cure shrinkage, low coefficient of thermal expansion and high strength. It aggressively bonds by chemical coupling to dentin, enamel, composites used in dentistry, porcelain and metal, such as stainless steel. It is a paste/paste formulation that is easy to mix. It is capable of rapid cure by exposure to room temperature and for more rapid cure, by exposure to light. In addition, though it contains a fluoride, which could be toxic when ingested in large dosages, it is biocompatible and safe to use on humans or other animals when applied topically.

Tenure® is a solvent based crosslinkable acrylic resin, provided as a solution/solution formulation. Its composition is described in U.S. Pat. No. 4,964,911, patented Oct. 27, 1990, and more effectively disclosed in U.S. Pat. Re 34,937, the disclosure of which is incorporated by reference. It is not an ionomer and does not release fluoride ion. It is less hydrophilic than Geristore®. It too is a crosslinkable resin. It contains a volatile solvent (typically acetone), which readily evaporates. After evaporation, a film of the resin rapidly cures in situ. Tenure® bonds by chemical coupling to dentin, enamel, porcelain, metal and the composites typically used in dentistry. It has been recommended for use with Geristore® in chemically bonding Geristore® to dentin or enamel.

Galan, Journal Of Esthetic Dentistry, Vol. 3, No. 6, (November/December 1991), describes the general use of Geristore® in the restoration of teeth and lesions both supra and subgingivally located.

M. Dragoo (unpublished) has used Geristore® in subgingival restorations of teeth to treat subgingival root resorption, split roots, endo perforation, tooth fracture, external root resorption and root coverage over previously restored and/or eroded root surfaces. He found the Geristore® aided in rebuilding biologic width, resulting in new tissue attachment, and minimized plaque induced gingivitis.

Body tissues are oftentimes subjected to undesirable afflictions such as irritation, decay or damage of bone or soft tissue. Irritation can be reflected in inflammation, decay can involve erosion and/or decomposition of tissue, and damage can be a wound or fracture. This invention involves topically treating mammalian, preferably human and domestic animal, tissue with a flexible wound dressing containing certain coating materials to decrease the impact of such afflictions. The device of the instant invention is a flexible film containing a high level of ionomer resin currently being marketed under Den-Mat's trade name Geristore®.

### SUMMARY OF THE INVENTION

One embodiment of the invention encompasses a process for enhancing the normal healing processes of a wound by providing at the area of a wound a flexible wound dressing comprising an ionomer resin.

Another embodiment of the invention encompasses a flexible wound dressing, wherein the flexible wound dressing further comprises silicone resin.

Another embodiment of the invention encompasses a flexible wound dressing, wherein the flexible wound dressing further comprises a pharmacological, healing or antibiotic agent.

A further embodiment of the invention encompasses a flexible wound dressing, wherein the flexible wound dressing is a film.

Yet another embodiment of the invention encompasses a flexible wound dressing comprising an adhesive.

Another embodiment of the invention encompasses a flexible wound dressing comprising a coloring agent.

A further embodiment of the invention encompasses a flexible wound dressing comprising a composition comprising SiO₂, P₂O5, Al2O3, Na2O, MO, and F, wherein MO is selected from the group consisting of BaO, BaO—CaO, BaO—SrO, and BaO—CaO—SrO; and a vinyl silicone material.

Yet another embodiment of the invention encompasses a flexible wound dressing comprising silicone.

Another embodiment of the invention encompasses methods of making a flexible wound.

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implicated in other compositions and methods, and that any such variation would be within such modifications that do not part from the scope of the present invention. Before explaining the disclosed embodiments of the present invention in detail, it is to be understood that the invention is not limited in its application to the details of any particular embodiment shown, since of course the invention is capable of other embodiments. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

The device of the instant invention is a flexible film containing a high level of ionomer-resin currently being marketed under Den-Mat's trade name Geristore®. Clinical trials have proven that cured Geristore®, when used as a dental restorative, exhibits biocompatible surface properties which preclude infections, and promote reattachment of gum tissue to the restored tooth. This gingival reattachment is quite unique, and therefore, the description of the present invention does not deal with formulation changes to Geristore® per se, but rather it deals with a method of making a flexible film which is filled with cured Geristore® made in situ. Such films should be usable for dressing all types of wounds for restoring the growth of skin tissue. Geristore® is a resin-ionomer composition with dual-cure capabilities (thermal and light), described in U.S. Pat. Nos.: 5,360,770; 5,876,743; 5,683,249; and 5,334,625. In normal use it cures rapidly into a very hard mass.

Additional therapeutic compositions that promote the wound healing process may be incorporated into the device and methods of the instant invention. For example, the device may include the incorporation of antimicrobial compositions, including but not limited to antifungal compositions, antibacterial compositions, antiviral compositions and antiparasitic compositions.

The present invention, both composition and methods, deals with the making of a silicone-rubber flexibillized Geristore® filled film by accurately timing the rapid blending of the two systems so that the highly dispersed A and B parts of Geristore® cure into very fine filler particles, just before the start of the crosslinking of the two-component silicone resin.

In order to make the film, the mixture of the four parts (two of Geristore®, two of silicone resin) is spread (“doctor-bladed”) directly onto a glass plate, or into the mesh of a fabric or foam.

### EXAMPLE 1

Preparation of Part 1:

In a small polypropylene beaker, mix 2.25 grams of Part A of Geristore® (comprising approximately equivalent amounts of an aromatic dimethacrylate oligomer and 2-hydroxyethyl methacrylate along with fillers, some initiators, and polymerization stabilizers), and 0.75 grams of the Part A of a commercially available vinyl silicone system (Silpak R-2458). After blending it thoroughly, add 0.0075 grams of 2-3% solution of a Platinum divinyltetramethyldisiloxane complex. Continue blending until the mixture is homogeneous.

Preparation of Part 2:

In a small polypropylene beaker mix 2.25 grams of the Part B of Geristore® (an aromatic dimethacrylate oligomer along with fillers, some photoinitiators and a chelating agent), and 0.75 grams of the Part B of Silpak R-2458. Blend mixture until homogeneous.

Prior to mixing Part 1 and Part 2, a 3 by 3 inch square thin cotton cloth is laid flat on a glass plate.

In a polypropylene beaker, equal portions of Part 1 and Part 2 (by weight) are stirred vigorously for 5 minutes. The blend is then transferred quickly onto the end of the fabric and then spread into the mesh of the fabric using a glass rod. This spreading should also be done on the other side of the fabric by flipping the fabric over and continuing the spreading. The spreading should be done quickly before the silicone cures into an unspreadable rubber.

### EXAMPLE 2

Preparation of Part 1:

In a small polypropylene beaker mix 1.403 grams of the Part A of Geristore® and 0.60 grams of the Part B of a commercially available vinyl silicone system (Silpak R-2438). Stir mixture until homogeneous.

Preparation of Part 2:

In a separate small polypropylene beaker mix 1.405 grams of the Part B of Geristore® and 0.603 grams of the Part A of Silpak R-2438. Stir until the mixture is homogeneous.

Before mixing Part 1 and Part 2, a piece of 40-mil open-cell foam sheet is laid on a glass plate.

In a polypropylene beaker, add 1.805 grams of Part 1 and 1.805 grams of Part 2 (preventing contact with each other) and then mix the two parts vigorously for 60 seconds. A portion of the blend is transferred quickly onto one end of the foam strip and spread into the mesh of the foam using a glass rod. The strip is then flipped over and the remaining portion of the blend is placed on this reverse side and spread until smooth. The spreading should be completed within 6 minutes. After about 4 minutes the sample (still slightly tacky) is placed into a light curing oven and exposed to light for about 10 minutes. The finished sample is flexible, soft, and virtually tack free.

While the invention has been described with reference to certain exemplary embodiments thereof, those skilled in the art may make various modifications to the described embodiments of the invention without departing from the scope of the invention. The terms and descriptions used herein are set forth by way of illustration only and are not meant as limitations. In particular, although the present invention has been described by way of examples, a variety of compositions and methods would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A flexible wound dressing comprising an ionomer resin.

2. The flexible wound dressing of claim 1, wherein the flexible wound dressing further comprises silicone resin.

3. The flexible wound dressing of claim 1, wherein the flexible wound dressing further comprises addition of healing or antibiotic agents.

4. The flexible wound dressing of claim 1, wherein the flexible wound dressing is a film.

5. The flexible wound dressing of claim 1, wherein the flexible wound dressing further comprises an adhesive.

6. The flexible wound dressing of claim 1, wherein the flexible wound dressing further comprises a coloring agent.

7. A method of making a flexible wound dressing comprising:
(a) providing a predetermined amount of an aromatic dimethacrylate oligomer, a 2-hydroxyethyl methacrylate, benzoyl peroxide and a polymerization stabilizer;
(b) providing a first predetermined amount of a vinyl silicone material;
(c) thoroughly mixing the compounds from step (a) and step (b) together to form a first substantially homogenous mixture;
(d) providing a predetermined amount of an aromatic dimethacrylate, a photoinitiator and a chelating agent;
(e) providing a second predetermined amount of a vinyl silicone material;
(f) thoroughly mixing the compounds from step (d) and step (e) together to form a second substantially homogenous mixture;
(g) thoroughly mixing the first and second mixtures together to form a third mixture; and
(h) applying the third mixture to a substrate to form a film to be used as a flexible wound dressing.

8. The method of claim 7, further comprising the step of adding a pharmacological, or antibiotic composition to any one of the mixtures.

9. The method of claim 7, further comprising the step of adding a coloring agent to any one of the mixtures.

10. The method of claim 7, further comprising the step of adding an agent that promotes hemostasis to any one of the mixtures.

11. A flexible wound dressing comprising:
a composition comprising SiO₂, P₂O5, Al2O3, Na2O, MO, and F, wherein MO is selected from the group consisting of BaO, BaO—CaO, BaO—SrO, and BaO—CaO—SrO, and a vinyl silicone material.

12. The flexible wound dressing of claim 11, further comprising an antibiotic.

13. The flexible wound dressing of claim 11, further comprising an analgesic.

14. The flexible wound dressing of claim 11, further comprising a coloring agent.

15. The flexible wound dressing of claim 11, further comprising a hemostasis-promoting agent.

16. The flexible wound dressing of claim 11, further comprising an adhesive.

17. A flexible wound dressing comprising:
a flexible substrate; and
a biocompatible material comprising a glass composition comprising SiO₂, P₂O5, Al2O3, Na2O, MO, and F, wherein MO is selected from the group consisting of BaO, BaO—CaO, BaO—SrO, and BaO—CaO—SrO; a vinyl silicone material, and a pharmacological, or antibiotic composition.

18. The flexible wound dressing of claim 17, wherein the pharmacological or antibiotic agent is a hemostasis promoting agent.

19. The flexible wound dressing of claim 17, wherein the pharmacological or antibiotic agent is an analgesic.
